(19) United States
(12) Patent Application Publication
(10) Pub. No.: US 2021/0236664 A1
(43) Pub. Date: Aug. 5, 2021

(54) ALPHA PARTICLE FORMULATIONS FOR TREATMENT OF SOLID TUMORS

(71) Applicant: The Johns Hopkins University, Baltimore, MD (US)

(72) Inventors: Jessie R. Nedrow, Baltmore, MD (US); Eleni Liapi, Baltimore, MD (US)

(21) Appl. No.: 17/050,088

(22) PCT Filed: Apr. 25, 2019
(86) PCT No.: PCT/US2019/029051

§ 371 Date: Oct. 23, 2020

Related U.S. Application Data
(60) Provisional application No. 62/662,733, filed on Apr. 25, 2018.

(51) Int. Cl. A61K 51/04; A61K 51/12; A61P 35/04

(52) U.S. Cl. CPC A61K 51/0482; A61K 51/1217; A61P 35/04; A61K 51/0402; A61K 51/0497

(57) ABSTRACT

Alpha-emitting LIPIODOL® emulsions and their use in treating cancer is disclosed. Radionuclides, including, but not limited to actinium-225 and bismuth-212/lead-212, are incorporated in LIPIODOL® emulsions. The alpha-emitting LIPIODOL® emulsions can be used to treat primary and metastatic liver cancer, including hepatocellular carcinoma (HCC), and metastatic diseases of the liver, as well as lymphatic cancers.

### BACKGROUND

Hepatocellular carcinoma (HCC) is a primary malignancy of the liver. Due to its indolent course, most patients afflicted with HCC have advanced and unresectable disease at the time of diagnosis and typically are offered only non-surgical palliative treatment options (Liapi, et al., 2007; Liapi, et al., 2010). The liver also is the most common site for metastatic disease for various types of cancer, including metastatic breast cancer.

Intra-arterial therapies are widely used for treatment of patients with HCC or metastatic liver cancer (Liapi, et al., 2007). The most commonly used intra-arterial therapies include transarterial embolization (TAE), chemoembolization (TACE), and radioembolization (TARE). TACE and TARE, in general, demonstrate higher survival rates than TAE in a 3-year window; however, TARE is considered less toxic than TACE (Yang, et al., 2012; Kennedy, et al., 2006; Llovet, et al., 2002; Maluccio, et al., 2008). The main side effect associated with these therapies is post-embolization syndrome, which has been suggested to be less pronounced when using a degradable embolizing agent, such as LIPIODOL® (Vogl, et al., 2007).

LIPIODOL®, also referred to ethiodized oil, is a poppy seed-based oil that can be used as a radio-opaque contrast agent. More particularly, as noted hereinabove, LIPIODOL® is used in chemoembolization applications as a contrast agent. LIPIODOL® also can be used in lymphangiography, i.e., the imaging of the lymphatic system. Due to its FDA-approved status, detailed information on its pharmacology, formulation and toxicity exists.

Alpha-particle radiopharmaceutical therapy (αRPT) provides cytotoxic agents that are considered impervious to conventional cellular resistance mechanisms, such as effusion pumps, signaling pathway redundancy, and cell cycle modulation (e.g., cell dormancy, G1/G0 or G2/M block) (Ballangrud, et al., 2001; Ballangrud, et al., 2000; Barendsen, et al., 1960a; Barendsen, et al., 1960b; Bloomer, et al., 1984a; Bloomer, et al., 1984b; Bloomer, et al., 1981; Humm, 1987; Humm, et al., 1993; Kassis, et al., 1986; Kozak, et al., 1986; Kurtzman, et al., 1988; Macklis, et al., 1988; McDevitt, et al., 2001; Raju, et al., 1991; Sgouros, et al., 1999; Ballangrud, et al., 2004; Imam, 2001; McDevitt, et al., 1998).

Alpha particle traversals through the DNA are highly damaging to cells. As few as one or two tracks through the nucleus can cause largely irreparable DNA double strand breaks, effectively killing the cell. Consequently, there is growing interest in α-particle emitters for cancer therapy (Rosenblat, et al., 2010; Sgouros, 2008; Milenic, et al., 2007). Pre-clinical studies have demonstrated promising results in αRPT. For example, in an animal model of bladder cancer it was found that 90% of mice survive >300 d following treatment with a ²¹³Bi-labeled antibody with reduced renal and genitourinary toxicity compared to treatment mitomycin C, which only had a 40% survival of mice (Pfost, et al., 2009). In a disseminated peritoneal disease model, ²¹²Pb-labeled antibody (²¹²Pb is parent to the α-emitter ²¹²Bi) demonstrated increased survival over untreated mice and in mice treated with gemcitabine (Milenic, et al., 2007). In clinical studies that have progressed beyond phase I, α-emitters have yielded significant survival results in adult leukemia (Rosenblat, et al., 2010; Rosenblat, et al., 2007), glioblastoma multiforme (Zalutsky, et al., 2008) and hormone-refractory metastatic prostate cancer (Nilsson, et al., 2007; Nilsson, et al., 2010), all cancers for which chemotherapy is ineffective. αRPT has demonstrated promising results both pre-clinically and clinically in treating metastatic and late-stage cancers, including cancers that have developed resistance to chemotherapy and receptor targeted therapies.

The potential of αRPT is further highlighted from clinical experience of the recently FDA-approved alpha-particle emitting agent, XOFIGO®, which has demonstrated significant success in patients with metastatic castrate-resistance prostate cancer. XOFIGO® is a bone-seeking α-emitting chelate whose radionuclide is preferentially incorporated into osteogenic sites including osteoclasts at the site of metastatic disease. The high linear energy transfer and very short (80 micron) range of alpha radiation results in potent cell kill to targeted cells and reduced toxicity to healthy cells and tissue, helping to minimize side effects. The common side effects of XOFIGO® include nausea, diarrhea, vomiting, and peripheral edema, which can be easily managed in patients receiving α-therapy treatment. XOFIGO® was approved based on the Alpharadin in Symptomatic Prostate Cancer (ALSYMPCA) trial that demonstrated a 2.8-month increased survival benefit in castration resistant prostate cancer patients. See “A Phase III Study of Radium-223 Dichloride in Patients With Symptomatic Hormone Refractory Prostate Cancer With Skeletal Metastases (ALSYMPCA),” NCT00699751. The success of XOFIGO® in the treatment of metastatic castrate resistant prostate cancer demonstrates that α-therapy is capable of treating metastatic prostate cancer, which is resistant to chemotherapy and anti-androgen receptor therapy (receptor targeted therapy).

LIPIODOL® accumulates and remains in the tumor while clearing out of normal liver tissue when injected via the hepatic artery, providing an excellent vehicle for the selective delivery of therapeutic radionuclides. β-emitting radionuclides have been explored and have demonstrated promising results. For example, HCC patients treated with ¹³¹I-LIPIODOL® vs. TACE had similar survival up to three years post-treatment; however, patients that had a portal vein thrombosis or more advanced disease demonstrated a significantly higher mean survival as compared to patients treated with TACE (Marelli, et al., 2009).

Non-LIPIODOL® based β-emitting RPT, such as the commercially available TheraSpheres®, have been developed using Yttrium-90 impregnated glass beads. The beads are infused using the hepatic artery, but unlike LIPIODOL® (bilobar infusion), only a single lobe can be infused at a time. Additionally, the direct uptake of LIPIODOL® into tumor cells vs. the accumulation of glass beads in arterioles results in a higher tumor radiation dose (Marelli, et al., 2009). Finally, patients having metastatic liver disease were imaged following administration of ¹³¹I-LIPIODOL® emulsions; the assessment of the distribution and retention of ¹³¹I-LIPIODOL® emulsions within tumors of varying sizes demonstrated that smaller metastases (<5 cm) gave the highest tumor to liver ratios (63/64:1) (Hind, et al., 1992).

The incorporation of ¹³¹I into LIPIODOL®, however, is costly and alternate β-emitting radionuclides have been explored. The chelation of ¹⁸⁸Re into a lipophilic chelator provided a more cost efficient compound for emulsion within LIPIODOL® for administration to HCC patients via an intra-arterial injection allowing accumulation within liver tumor sites. Treatment with ¹⁸⁸Re-LIPIODOL® demonstrated a 25% objective response rate (ORR) (3% complete remission), and 53% of patients had stable disease. Toxicity (grade 3 or 4) was limited to hepatic toxicity (12%), and hematologic toxicity (3%). Hepatic toxicity is indicative of non-specific uptake in normal liver cells and/or decay of the β-particle outside the tumor site. ¹⁸⁸Re-LIPIODOL®, a chelate-LIPIODOL® emulsion demonstrated positive results in clinical trials (similar results were seen with ¹³¹I-LIPIODOL®) the potential to increase the ORR by increasing the dose of β-emitting LIPIODOL® may be limited by increased hematologic toxicity. In addition, the low energy transfer of the emitted β-particle introduces the ability of the remaining tumors to develop resistance mechanisms further limiting the treatment options for patients.

The expanding use of TAE, TACE, and TARE required the development of imaging protocols to assess the shunting of these treatments to the lungs, which are known to occur in liver tumors. ^{99m}Tc-MAA (macroaggregated albumin) provided a SPECT imaging agent to help determine the percentage of lung shunting in patients, necessary for limiting potential toxicity to the lungs (Leung, et al., 1994; Ahmadzadehfar, et al., 2010). While ^{99m}Tc-MAA has been crucial in identifying the percentage of lung shunting in patients, its short half-life (6.0 hours) limits its use as a surrogate-imaging agent for dosimetric evaluation of long-lived targeted radiotherapeutics. Indium-111 provides a longer-lived SPECT radionuclide (T_{1/2}=2.8 days) that is capable of serving as a surrogate-imaging agent for therapeutic nuclides, such as, the β-emitting radionuclides Yttrium-90 and Luteticium-177, as well as the α-emitting radionuclide Actinium-225. surrogate imaging-agents can provide pharmacokinetics and clearance properties of their therapeutic counterparts, allowing for more accurate absorbed doses to the tumor, as well as non-target organs including the lungs. Accurate absorbed doses help better estimate the overall dose administered, providing the maximum tolerated dose while limiting toxicity.

### SUMMARY

In some aspects, the presently disclosed subject matter is directed to a formulation comprising a chelator coupled to a lipophilic alkyl side chain and an emulsifying agent comprising poppy seed oil, e.g., LIPIODOL®.

In particular aspects the chelator is selected from the group consisting of: H₂macropa, 2-(4-isothiocyanatobenzyl)-1,4,7,10,13,16-hexaazacyclohexadecane-1,4,7,10,13,16-hexaacetic acid (HEHA-NCS), 1,4,7,10,13,16-hexaazacyclohexadecane-N,N′,N″,N′″,N″″,N′″″-hexaacetic acid (HEHA),

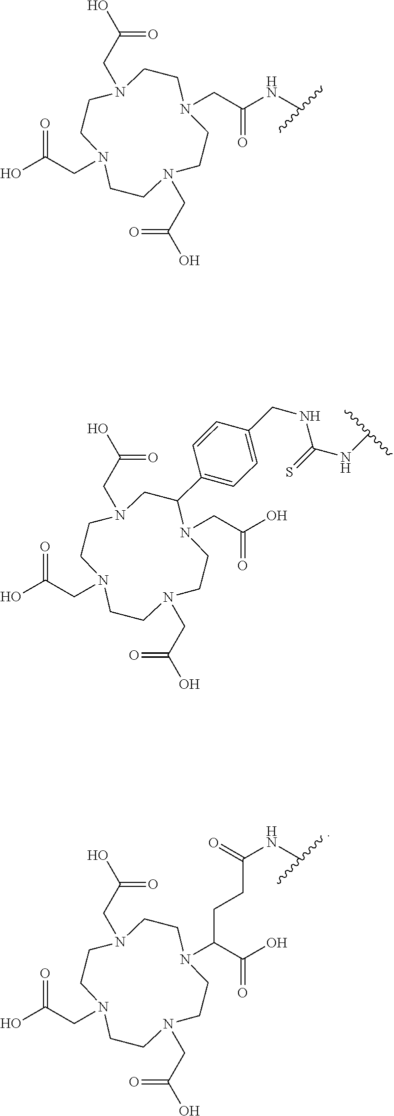 [SMILES: *NC(=O)CN1CCN(CC(=O)O)CCN(CC(=O)O)CCN(CC(=O)O)CC1 ; *NC(=S)NCc1ccc(C2CN(CC(=O)O)CCN(CC(=O)O)CCN(CC(=O)O)CCN2CC(=O)O)cc1 ; *NC(=O)CCC(C(=O)O)N1CCN(CC(=O)O)CCN(CC(=O)O)CCN(CC(=O)O)CC1]

In certain aspects, the alkyl side chain comprises a fatty acid. In yet more certain aspects, the alkyl side chain is selected from the group consisting of:

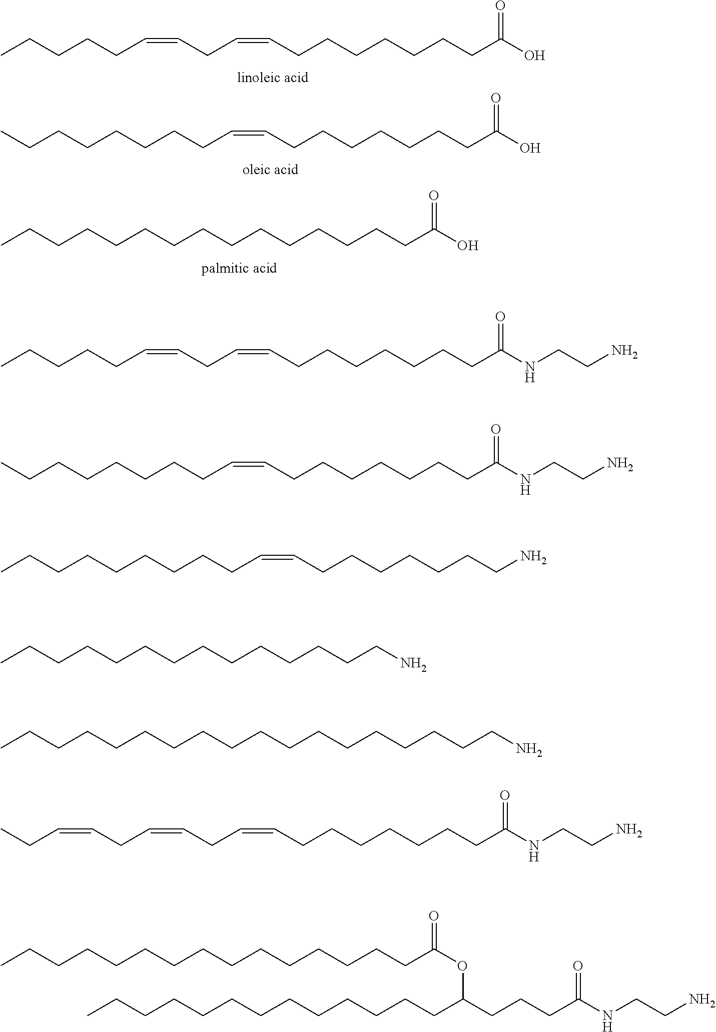

In further aspects, the formulation comprises an alpha-emitting radionuclide chelated to the chelator through one or more coordinate bonds. In particular aspects, the alpha-emitting radionuclide is ²²⁵Ac.

In yet other aspects, the formulation comprises a radionuclide suitable for use in single-photon emission computed tomography (SPECT) imaging or positron emission tomography (PET) imaging, wherein the radionuclide is chelated to the chelator through one or more coordinate bonds. In particular aspects, the radionuclide is indium-111 (¹¹¹In).

In some aspects, the presently disclosed formulation can be used to treat cancer. In certain aspects, the cancer is a primary liver cancer. In yet more certain aspects, the primary liver cancer comprises hepatocellular carcinoma (HCC). In other aspects, the cancer is a metastatic cancer. In yet other aspects, the cancer comprises a lymphatic cancer.

In other aspects, the presently disclosed subject matter provides a method for imaging a cancer in a subject, the method comprising administering to the subject the formulation as described hereinabove and taking an image. In particular embodiments, the imaging is SPECT imaging.

Certain aspects of the presently disclosed subject matter having been stated hereinabove, which are addressed in whole or in part by the presently disclosed subject matter, other aspects will become evident as the description proceeds when taken in connection with the accompanying Examples and Drawings as best described herein below.

### BRIEF DESCRIPTION OF THE DRAWINGS

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 shows CT volumetric calculations for intratumor LIPIODOL® deposition in the VX2 rabbit liver model;

FIG. 2 shows the chemical structure of DOTAGA-tetradecylamine (DOTAGA-TDA);

FIG. 3A, FIG. 3B, and FIG. 3C show CT with contrast dye of rabbit with a VX2 tumor prior to injection of ²²⁵Ac-DOTAGA-TDA: (FIG. 3A) Hepatic Artery; (FIG. 3B) VX2 tumor; and (FIG. 3C) Hepatic Artery branch to VX2 tumor;

FIG. 4 shows representative structures of DOTA derivatives and representative side chain modifiers based on poppy seed oil;

FIG. 5 shows alpha camera imaging of VX2 tumor treated with intra-arterial injection of α-RPT-LIPIODOL® emulsion, at the 24-hr time point, demonstrating preferential uptake of the emulsion at the tumor rim; and

FIG. 6A and FIG. 6B demonstrate the delivery of ¹¹¹In-labeled LIPIODOL® emulsion to tumor in mouse model. FIG. 6A and FIG. 6B show uptake of ¹¹¹In-DOTA-TDA-LIPIODOL® within the tumor compared to ¹¹¹In-DOTA-TDA alone after (FIG. 6A) 24 hours (209±39.7% ID/g vs. 103±65.0% ID/g;p≤0.05) and (FIG. 6B) after 48 hours (216±145% ID/g vs. 20.0±15.9% ID/g;p≤0.0001).

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Alpha Particle Formulations for Treatment of Solid Tumors

Embolization-based treatments, such as TACE and TARE, have shown promising results in the treatment of HCC and metastatic liver disease. LIPIODOL®-based embolization treatments have demonstrated a reduced risk of post-embolization syndrome and higher radiation dose to the tumor for TARE treatments. TARE has been limited, however, to β-emitting radionuclides, which are less potent than α-emitting radionuclides. Furthermore, the long range of β-particles exposes normal tissues to an unnecessary radiation dose resulting in increased toxicity. Limiting the dose to minimize toxicity, however, results in inadequate dosing to tumors, potentially allowing the tumors to develop resistance to radiotherapy. These limitations of β-emitting radionuclides highlight the need to develop αRPT-based therapeutic agents that are highly damaging over a short range to cancer cells and impervious to resistance.

Accordingly, the presently disclosed subject matter provides αRPT-LIPIODOL® agents and their use for treating primary and metastatic liver cancer, including hepatocellular carcinoma (HCC), and metastatic diseases of the liver, as well as lymphatic cancers. The presently disclosed agents are capable of accumulating in primary and metastatic liver tumors with minimal accumulation in normal organ tissues, thereby providing highly potent, targeted α-RPT agents with reduced side effects. Further, the presently disclosed α-RPT emulsions are capable of delivering radionuclides suitable for imaging, including single-photon emission computed tomography (SPECT) imaging, for targeted image-guided treatment of primary and metastatic liver cancer. For example, the delivery of a radionuclide suitable for use with SPECT imaging, e.g., indium-111 (¹¹¹In), to primary and metastatic liver tumors provides a companion-imaging agent for identifying patients who will benefit from this targeted treatment, as well as monitor treatment progress. Further, low-photon imaging protocols centered on ²²⁵Ac-labeled αRPT emulsions supports the development of a single theranostic agent for the treatment and monitoring of primary and metastatic liver tumors. Accordingly, the presently disclosed subject matter serves as a foundation for developing imaging protocols for ²²⁵Ac-labeled αRPT, potentially eliminating the need for companion-imaging agents. Thus, the presently disclosed ²²⁵Ac-labeled αRPT emulsions could potentially be capable of targeted imaging and treatment of primary and metastatic liver tumors.

Accordingly, in some embodiments, the presently disclosed subject matter provides a formulation comprising a chelator coupled to a lipophilic alkyl side chain and an emulsifying agent comprising poppy seed oil. In particular embodiments, the poppy seed oil is LIPIODOL® (Guerbet LLC, Princeton, N.J., United States). LIPIODOL® is an FDA-approved radiopaque agent comprising a variety of oils, including, but not limited to, the ethyl esters of fatty acids of poppy seed oil. Poppy seed oil is roughly about 56% to about 69% linoleic acid, about 16% to about 20% oleic acid, and about 11% to about 16% palmitic acid. In certain embodiments of the presently disclosed formulations, the poppy seed oil comprises about 50% to about 75% linoleic acid, including about 50%, 55%, 60%, 65%, 70%, and 75% linoleic acid; about 10% to about 30% oleic acid, including about 10%, 15%, 20%, 25%, and 30% oleic acid; and about 5% to about 25% palmitic acid, including about 5%, 10%, 15%, 20%, and 25% palmitic acid. In other embodiments, the poppy seed oil comprises about 55% to about 70% linoleic acid, about 15% to about 20% oleic acid, and about 10% to about 20% palmitic acid.

In some embodiments, the chelator is selected from the group consisting of:

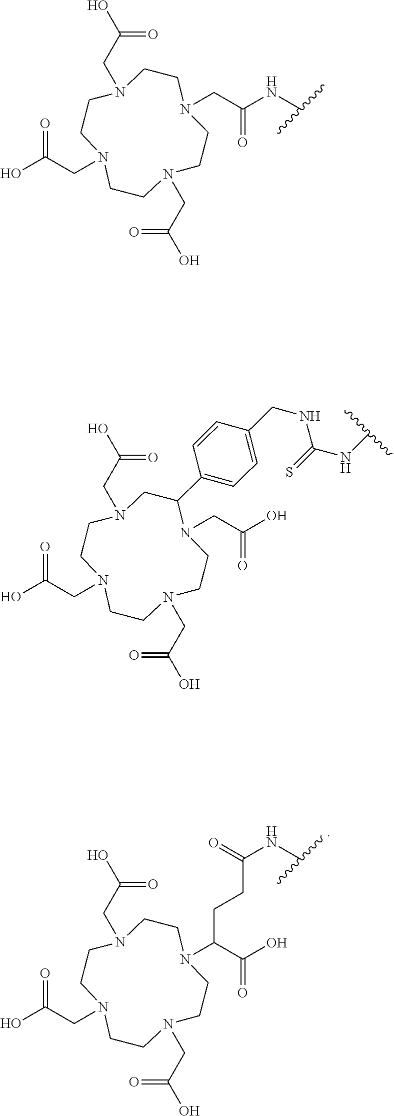 [SMILES: *NC(=O)CN1CCN(CC(=O)O)CCN(CC(=O)O)CCN(CC(=O)O)CC1 ; *NC(=S)NCc1ccc(C2CN(CC(=O)O)CCN(CC(=O)O)CCN(CC(=O)O)CCN2CC(=O)O)cc1 ; *NC(=O)CCC(C(=O)O)N1CCN(CC(=O)O)CCN(CC(=O)O)CCN(CC(=O)O)CC1]

One of ordinary skill in the art would appreciate that other chelators known in the art suitable for chelating an alpha-emitting radionuclide or a radionuclide suitable for use in SPECT or PET imaging would be suitable for use in the presently disclosed formulations, including, but not limited to H₂macropa, (see, Theile, N. A., et al., An Eighteen-Membered Macrocyclic Ligand for Actinium-225 Targeted Alpha Therapy, Angewandte Chemie, 65(46), 14712-14717 (2017)); 2-(4-isothiocyanatobenzyl)-1,4,7,10,13,16-hexaazacyclohexadecane-1,4,7,10,13,16-hexaacetic acid (HEHA-NCS) (see Chappell et al, Synthesis, Conjugation, and Radiolabeling of a Novel Bifunctional Chelating Agent for 225Ac Radioimmunotherapy Applications, Bioconjugate Chem., 2000, 11 (4), pp 510-519), and 1,4,7,10,13,16-hexaazacyclohexadecane-N,N′,N″,N′″,N″″,N′″″-hexaacetic acid (HEHA) (see, U.S. Pat. No. 6,696,551B1 to Brechbiel, M. W., et al., for ²²⁵Ac-HEHA and related compounds, methods of synthesis and methods of use, issued Feb. 24, 2004, which is incorporated herein by reference in its entirety.

In some embodiments, the lipophilic side chain comprises a fatty acid. In particular embodiments, the fatty acid is selected from a saturated fatty acid and an unsaturated fatty acid. In yet more particular embodiments, the saturated fatty acid is selected from the group consisting of caprylic acid, capric acid, lauric acid, myristic acid, palmitic acid, stearic acid, arachidic acid, behenic acid, lignoceric acid, and cerotic acid. In other embodiments, the unsaturated fatty acid is selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid.

In particular embodiments, the alkyl side chain is selected from the group consisting of:

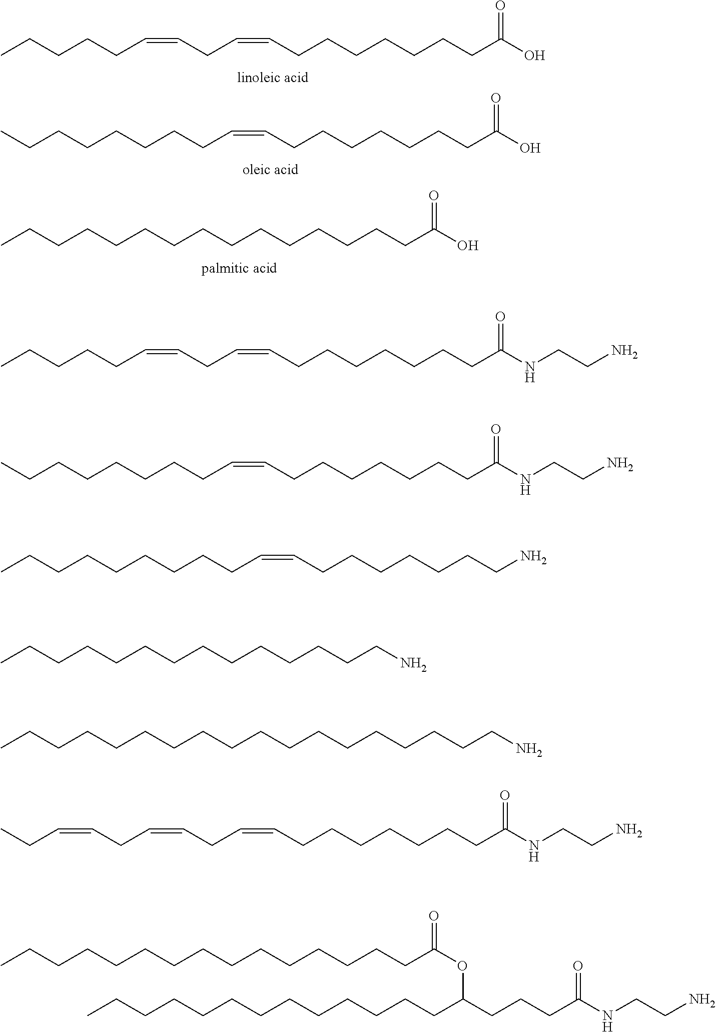

The chelator can be coupled to the alkyl side chain through an amine-reactive pendant group on the carbon backbone of the alkyl side chain or through an activated carboxylic acid of the alkyl side chain.

In certain embodiments, the chelator coupled to the lipophilic alkyl side chain has the following chemical structure:

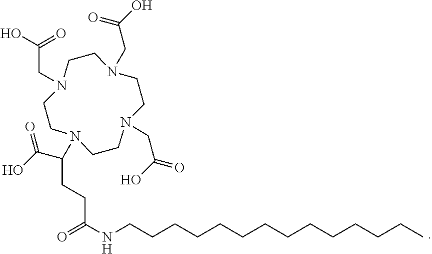 [SMILES: CCCCCCCCCCCCCCNC(=O)CCC(C(=O)O)N1CCN(CC(=O)O)CCN(CC(=O)O)CCN(CC(=O)O)CC1]

As one of ordinary skill in the art would appreciate, the chelator can chelate an alpha-emitting radionuclide through one or more coordinate bonds. In certain embodiments, the alpha-emitting radionuclide is selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb) Thorium-227 (²²⁷Th), and Terbium-149 (¹⁴⁹Tb),In particular embodiments, the alpha-emitting radionuclide is ²²⁵Ac.

In other embodiments, the formulation further comprises a radionuclide suitable for use in single-photon emission computed tomography (SPECT) or PET imaging, wherein the radionuclide is chelated to the chelator through one or more coordinate bonds. In particular embodiments, the radionuclide suitable for use in SPECT imaging is selected from the group consisting of indium-111 (¹¹¹In), technetium-99m (^{99m}Tc), thallium-201 (²⁰¹Tl), terbium-155 (¹⁵⁵Tb), gallium-68 (⁶⁸Ga), copper-64 (⁶⁴Cu), and zirconium-89 (⁸⁹Zr).

In other embodiments, the presently disclosed subject matter provides a method for treating cancer in a subject in need of treatment thereof, the method comprising administering to the subject an effective amount of the presently disclosed formulation comprising an alpha-emitting radionuclide. In certain embodiments, the cancer comprises a primary liver cancer. In more certain embodiments, the primary liver cancer comprises hepatocellular carcinoma (HCC). In other embodiments, the cancer comprises a metastatic cancer. In certain embodiments, the metastatic cancer comprises metastatic breast cancer. In yet other embodiments, the cancer comprises a lymphatic cancer. In particular embodiments, the formulation is delivered intra-arterially.

In other embodiments, the presently disclosed subject matter provides a method for imaging a cancer in a subject, the method comprising administering to the subject the presently disclose formulation comprising a radionuclide suitable for use with SPECT imaging and taking an image. Accordingly, in particular embodiments, the imaging is SPECT imaging.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

In general, the “effective amount” of an active agent or drug delivery device refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Delivery of ²²⁵Ac-Labeled LIPIODOL® Emulsion to Tumor in Rabbit Model

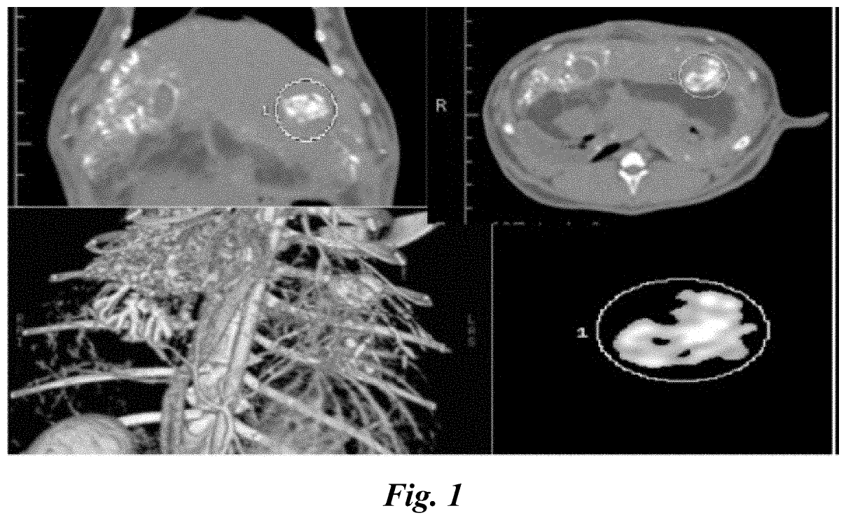

FIG. 1 demonstrates the prior use of the technical rabbit VX2 model of liver cancer and the use of various embolic therapies, including LIPIODOL®. See Attaluri, A., et al., 2016; Gholamrezanezhad, A., et al., 2016; Lee, K. H., et al., 2009a; Lee, K. H., et al., 2009b; Lee, K. H., et al., 2008; Lee, K. H., et al., 2008; Lee, K. H., et al., 2010; Liapi, E., et al., 2011; Vali, M., et al., 2007; Vali, M., et al., 2008; and Vossen, J. A., et al., 2009. Typically, rabbit tumors have a maximal axial diameter of about 1.5 cm to about 2 cm before treatment and if untreated, tumors dramatically increase in size within a week to reach a diameter of about 3 cm to about 3.5 cm. At this time, rabbits also exhibit lung metastases. Lee, K. H., et al., 2009a; Lee, K. H., et al., 2009b; Vali, M., et al., 2007; Parvinian, A., 2014.

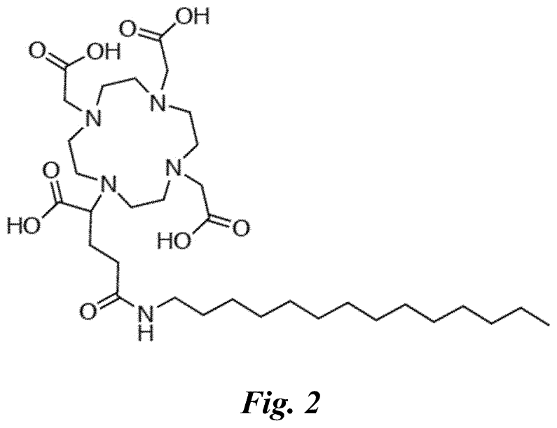

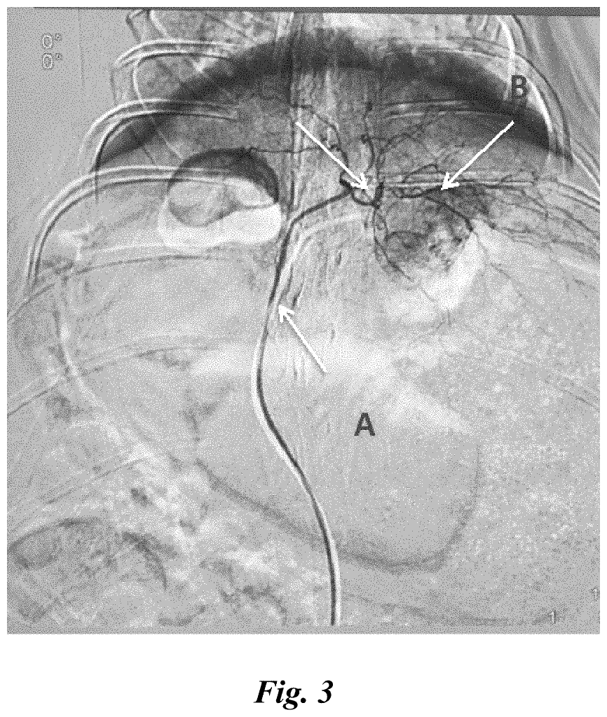

The feasibility of delivering the presently disclosed ²²⁵Ac-labeled LIPIODOL® emulsion via the intrahepatic artery (Rabbit Model) was determined. The successful synthesis of DOTAGA-tetradecylamine (TDA) (FIG. 2) was confirmed by NMR and mass spectrometry. DOTAGA-TDA was then labeled with ²²⁵Ac, having greater than >90% radiolabeling yield and a specific activity of 2.5 μCi/μg. The resulting complex, ²²⁵Ac-DOTAGA-TDA, was emulsified in LIPIODOL® without further purification. The ²²⁵Ac-DOTAGA-TDA-LIPIODOL® emulsion was injected via the intrahepatic artery of a New Zealand Rabbit that had been implanted with a VX2 tumor (FIG. 3B). A branch (FIG. 3C) of the intrahepatic artery (FIG. 3A) was supplying the VX2 tumor, allowing for a targeted delivery of the ²²⁵Ac-DOTAGA-TDA-LIPIODOL® emulsion. Three hours post-injection the rabbit was euthanized and an ex vivo biodistribution was performed (Table 1).

TABLE 1
Ex vivo Biodistribution of ²²⁵Ac-DOTAGA-
TDA-LIPIODOL ® Emulsion in
Rabbit VX2 Model of Liver Cancer

| Organ | Specific Activity (Bq/g) |
|---|---|
| Blood | 6.27 |
| Liver | 16.21 |
| Tumor | 207.05 |
| Right Kidney | 32.79 |
| Spleen | 2.25 |
| Lung | 7.29 |
| Gall Bladder (Empty) | 41.15 |
| Muscle | 2.26 |
| Heart | 3.83 |

The ²²⁵Ac-DOTAGA-TDA-LIPIODOL® emulsion accumulated within the tumor while clearing out of the normal tissue, including the liver. The ²²⁵Ac-DOTAGA-TDA-LIPIODOL® emulsion had a tumor to liver ratio of 13:1. Furthermore, the tumor had the highest activity per gram in the organs evaluated.

The VX2 liver tumor model is commonly utilized to investigate preclinical treatments delivered through the hepatic artery. The rabbit VX2 tumor model was developed to study solid human cancers, including liver tumors. Briefly, the VX2 model isolates fragments of a VX2 tumor grown on the hind leg of the rabbit and implants the fragments of the VX2 tumor into the liver, where it forms a solid liver tumor. Lee, K. H., et al., 2008; Kidd, J. G., et al., 1940. Due to the large vasculature of the rabbit, experimental treatments can be administered via the hepatic artery to evaluate their effectiveness to accumulate and treat a solid liver tumor. Josefsson, A., et al., 2017. The VX2 model disclosed herein will help access the therapeutic efficacy of the presently disclosed αRPT-LIPIODOL® emulsions against a solid tumor model to access the delivery of αRPT-LIPIODOL® via the intra-hepatic injection method.

In contrast, orthotropic rat models have been utilized to investigate targeted treatment to liver tumors by administering treatment via the portal vein. The blood supply to normal liver mainly stems from the portal artery (70-80%), while the hepatic artery predominantly supplies hepatic tumors with little or no blood supply from the portal vein. Hobbs, R. F., et al., 2011. The administration of treatment via the portal vein would alter the therapeutic efficacy, as well as the toxicity of the presently disclosed αRPT-LIPIODOL® emulsions, due to the treatment being directed to the normal liver and not the tumor.

In other embodiments, mouse models can be used to assess the initial retention and penetration, as well as the therapeutic efficacy of the presently disclosed αRPT-LIPIODOL® emulsions in a HCC model (HEP2G), as well as a breast cancer model (4Tl). Mouse models can be used to evaluate the αRPT-LIPIODOL® emulsions in subcutaneous tumors injected intratumorally, providing an inexpensive animal model to initially evaluate αRPT-LIPIODOL® emulsions.

### Example 2

### Alpha Emitting and Companion SPECT Imaging LIPIODOL® Emulsions

As noted hereinabove, LIPIODOL® is an FDA-approved radiopaque agent comprising a variety of oils, including, but not limited to, the ethyl esters of fatty acids of poppy seed oil. Poppy seed oil is roughly about 56% to about 69% linoleic acid, about 16% to about 20% oleic acid, and about 11% to about 16% palmitic acid. Azcan et al., 2004. The introduction of fatty acid alkyl side chains to chelators can be accomplished through amine coupling reactions. Previous work exploring ¹⁸⁸Re-LIPIODOL® emulsions, as well as the presently disclosed ²²⁵Ac-DOTAGA-TDA-LIPIODOL® emulsions, demonstrated that the incorporation of tetradecylamine to a chelator provides agents that could be emulsified in LIPIODOL® for delivery to liver tumors. Further evaluations of derivatives of fatty acid side chains have the potential to provide αRPT-LIPIODOL® emulsions with superior tumor retention and penetration.

Accordingly, a library of αRPT agents can be synthesized by modifying components of poppy seed oil to present the DOTA or DOTAGA chelator for radiolabeling with ¹¹¹In (SPECT imaging) and ²²⁵Ac (targeted alpha therapy). αRPT agents that possess the following characteristics are suitable for use with the presently disclosed subject matter: (1) a partition coefficient_{[Octanol/LIP1ODOL®]} greater than two; (2) radiolabeling yields greater than 95%; and (3) high bench-top and serum stability (>95% for 3 days). Base on initial evaluations (e.g., partition coefficients, radiolabeling yields, and stability) compounds can be selected for in vivo experiments to evaluate tumor retention (SPECT imaging and biodistributions) and penetration (α-camera imaging). The previous synthesized DOTAGA-TDA and its radiolabeled derivatives will serve as a standard for comparison. Optimized scaffolds for ²²⁵Ac, and ¹¹¹In will be determined by superior radiolabeling efficiency, stability, and tumor retention and penetration.

### Example 3

### Synthesis of DOTA(GA)-alkyl Chelators

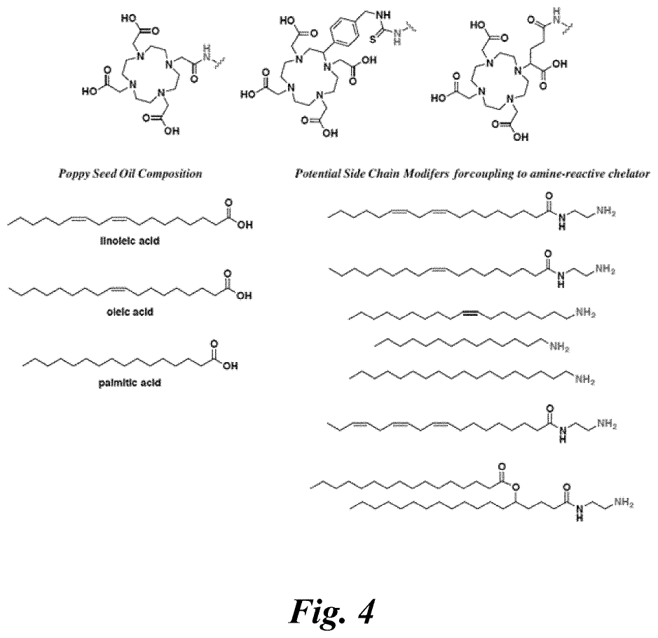

A library of lipophilic DOTA(GA) chelators for the incorporation of α-Lipophilicity can be introduced to the DOTA(GA) chelator, which is capable of complexing ²²⁵Ac and ¹¹¹In, by introducing alkyl side chains based on the fatty acids of poppy seed oil. The alkyl side chains can be coupled to the DOTA(GA) chelator through an amine-reactive pendant group on the carbon backbone or through an activated carboxylic acid (FIG. 4). The resulting compounds can be purified and then confirmed by NMR and mass spectrometry.

### Example 4

### Radiolabeling of DOTA(GA)-alkyl Chelators

Radiolabeling can be performed as routinely done for both ¹¹¹In and ²²⁵Ac. For example, DOTAGA-TDA (20 μg) was labeled with 3 MBq of ²²⁵Ac in 3M sodium acetate (pH=5.5) at 95° C. for 30 minutes with radiolabeling yields >90%. Conditions, such as buffer concentration and composition, length of time, and pH can be adjusted to optimize radiolabeling yields for the individual DOTA(GA)-derivatives.

### Example 5

### Determination of Partition Coefficients

Partition coefficients will be as previously described with some minor modifications. Decristoforo, C., et al., 2008. Octanol and saline (pH=7.4) will be used to assess the p-value of the proposed compounds. Briefly, the radiolabeled ¹¹¹In- and ²²⁵Ac-labeled DOTA(GA)-alkyl chelates will be vortexed with octanol and saline then centrifuged to separate layers. Aliquots (3) from each layer will be removed and counted. The p-value will be determined using the following equations:

P_{oct/saline}=Log([_{activity}]_{octanol}/[_{activity}]ₛₐₗᵢₙₑ).

### Example 6

### LIPIODOL® Retention

To assess the ability of the compounds to remain emulsified in LIPIODOL®, partition coefficient experiments will be performed using LIPIODOL® and saline (pH=7.4). The p-value will be determined as described for octanol and saline, but using LIPIODOL® in place of octanol:

(P_{LIPIODOL®/saline}=Log([_{activity}]_{LIPIODOL®}/[activity]ₛₐₗᵢₙₑ).

Furthermore, p-values will be determined over a 15-day window to assess the developed ¹¹¹In- and ²²⁵Ac-DOTA(GA)-alkyl chelates' ability to remain within LIPIODOL®.

### Example 7

### Stability of DOTA(GA)-Alkyl Chelates

Bench-top and serum stability will be performed as previously described. Lub-de Hooge, M. N., et al., 2004; Nedrow, J. R., et al., 2014; Beaino, W., et al., 2015. Briefly, radiolabeled DOTA(GA)-alkyl chelates will be placed in either saline (pH=7.4) or human serum. Stability of the developed radiopharmaceuticals, both bench top and serum stability, will be evaluated by HPLC over a 15-day window. The resulting lipophilic chelators will be radiolabeled with the proposed radionuclides in high radiochemical purity and extracted with LIPIODOL® to form α-emitting LIPIODOL® emulsions for evaluation. Superior radiolabeling efficiency, stability, and LIPIODOL® retention will determine a single optimized scaffold for ²²⁵Ac-labeled LIPIODOL® emulsions.

### Example 8

### Tumor Retention

The selected ¹¹¹In- and ²²⁵Ac-DOTA(GA)-alkyl chelates will be evaluated initially in vivo by SPECT imaging and biodistributions for tumor retention. The radiolabeled chelates will be vortexed with LIPIODOL®, centrifuged, and the LIPIODOL® emulsion will be extracted and prepared for injection. SPECT imaging and biodistributions will be performed as previously described on Nu/Nu mice (4-6 weeks) bearing HEP2G tumors and Balb/c mice bearing 4T1 tumors at the following time points: 2 h; 1 day, 3 days, 6 days, 9 days, 12 days, and 15 days. Tumor growth will be monitored by calipers to factor in tumor growth or regression due to the length of time monitoring tumor retention.

### Example 9

### SPECT Imaging

Intratumoral injections of the ¹¹¹In-DOTA(GA)-alkyl emulsified in LIPIODOL® or without LIPIODOL® will be administered to tumor-bearing mice (n=3/group). Tumor uptake and retention will be determined through analysis of SPECT images as compared to a known standard.

### Example 10

### Ex vivo Biodistributions

Intratumoral injections of the ²²⁵Ac-DOTA(GA)-alkyl emulsified in LIPIODOL® will be administered to tumor-bearing mice (n=5/group). Tumor uptake will be calculated as % ID/g as determined by a diluted standard. In addition, administration of the un-emulsified ²²⁵Ac-DOTA-alkyl will be evaluated at 6 days post-injection.

### Example 11

### Dosimetric Analysis

Data collected from In-111 and Ac-225 SPECT imaging and ex vivo biodistributions will be utilized to calculate the absorbed dose to the tumor, as well as clearance organs. Previously developed methods that have established the microscale distribution of alpha emissions to account for the short range and very localized energy deposition of alpha emitters will be utilized. These methods are necessary to adequately correlate dose to biological outcome, as whole organ average dosimetry often fails for α-particles. The principle has been described previously and rests on the macro-to-micro principle: by measuring whole organ and small scale activities (including α-emitting daughters) distribution over time ex vivo in a same organ, an organ- and radiopharmaceutical-specific schema for apportioning whole organ time-integrated activity to sub-organ compartments is established. Hobbs, et al., 2012. Sub-organ S-values established using geometrical shapes for sub-organ anatomy and GEANT4 Monte Carlo are then applied to convert sub-organ time-integrated activity for all to doses.

This method has been proven to be reliable for the kidney in the context of ²²⁵Ac-7.16.4, HER2-targeted therapy of metastatic breast cancer, Josefsson, A., et al., 2016, and others have been developed as required (salivary glands, thymus, bone, and the like). Josefsson, A., et al., 2016; Hobbs, R., et al., 2017; Hobbs, R. F., et al., 2012; Josefsson, A., et al., 2017. Depending on the organ and radiopharmaceutical, small-scale modeling may or may not be required. To date, lung and liver models have not proven necessary, although a small-scale model exists for Y-90 TheraSpheres®. Hogberg, J., et al., 2015. A tumor model for efficacy also exists, Hobbs, R. F., 2011; tumor dosimetry is more delicate as every tumor will have a different distribution. Nevertheless, information about uptake, diffusion, and penetration from ex vivo alpha-camera images can be generalized to substantially improve predictability versus average organ dose values.

### Example 12

### Penetration

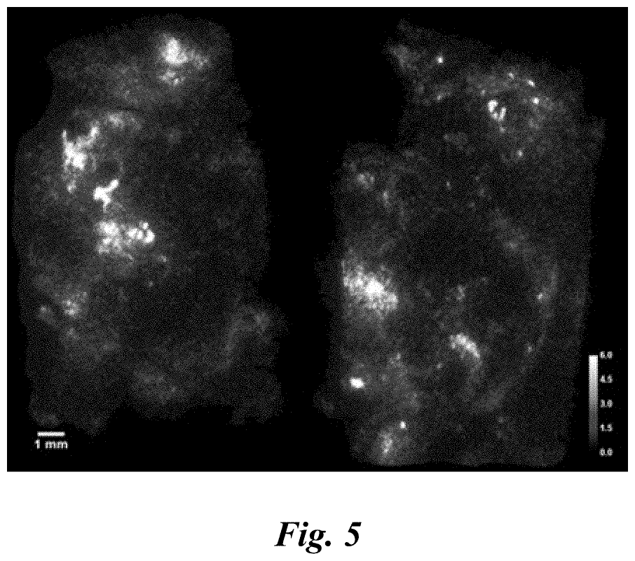

Penetration will be evaluated by 3D multi-photon confocal microscopy and α-camera autoradiography as previously performed. See, for example, FIG. 5. Rabbits with be imaged prior to the time points in the ex vivo biodistribution with focus on the tumor, liver, lungs, spleen, and kidneys. In other embodiments, following procedures described for ex vivo biodistribution for described, mice (n=3) will be euthanized and the tumor removed and prepped for α-camera imaging. Adjacent slices will be prepared on a slide for DAPI staining. Images will be overlaid for visual analysis of spheroid penetration. Depth of penetration will be determined by analysis of the resulting alpha tracks through the spheroid.

### Example 13

### CT Volumetry

Volumetric measurements of tumors and intra-tumor ²²⁵Ac-DOTA(GA)-alkyl emulsified in LIPIODOL® uptake (CT volumetry) will be obtained on contrast enhanced computed tomography (CECT) scans and non-contrast enhanced CT (NCECT) scans. For tumor volume calculations, the appropriate CT phase will be identified, for maximal depiction of tumor enhancement. Volumetric analysis will be based on a pixel-threshold algorithm. A region of interest (ROI) will be first selected from normal tissue and the ROI histogram of pixel attenuation will then be generated and inspected. A second ROI will then be generated within the region of intra-tumor ²²⁵Ac-DOTA(GA)-alkyl emulsified in LIPIODOL® deposition and a threshold cutoff value that best distinguishes ²²⁵Ac-DOTA(GA)-alkyl emulsified in LIPIODOL® from adjacent unenhanced normal tissue will be determined. Volumes will be then generated by manual or automatic segmentation based in the cutoff values defined from the above ROIs. Scans will be reviewed side by side and any accumulation of ²²⁵Ac-DOTA(GA)-alkyl emulsified in LIPTODOL® in non-tumorous tissues will not be taken into account for these measurements. For each tumor, ²²⁵Ac-DOTA(GA)-alkyl emulsified in LIPIODOL® retention will be defined as the ratio of the intra-tumor ²²⁵Ac-DOTA(GA)-alkyl emulsified in LIPIODOL® volume (L) to the tumor (T) volume (L/T). Since ²²⁵Ac-DOTA(GA)-alkyl emulsified in LIPIODOL® deposition in non-tumorous tissues will not be taken into account for these measurements, the highest possible ratio assigned can be 1. ²²⁵Ac-DOTA(GA)-alkyl emulsified in LIPIODOL® distribution into healthy tissues will be recorded and qualitatively measured, as described above.

Following procedures described for ex vivo biodistribution for described, sections of the tumor and normal organs will be removed and prepped for α-camera imaging. The resulting data will be compiled alongside the ex vivo biodistribution data to assess the potential of using CT to provide a dosimetric model for αRPT-LIPIODOL® emulsion.

### Example 14

### Statistical Analysis

For comparison between compounds, 5 mice in each group allow a detection of 15% difference in distribution with an alpha of 0.05 and a power of 0.96 using a one-way ANOVA. This difference in distribution is adequate to identify significant differences in organ distribution caused by tumor presence or compound differences.

### Example 15

### Determination of the Therapeutic Efficacy of α-Emitting LIPIODOL® Emulsions in Animal Models in Relation to Tumor Penetration and Retention

The therapeutic efficacy of the presently disclosed αRPT-LIPIODOL® emulsions will be initially determined in a subcutaneous mouse model of HCC (HepG2) and breast cancer model (4T1) (intratumoral injections). SPECT imaging will be performed using the companion-imaging agent (¹¹¹In-labeled DOTA(GA)-alkyl-chelate) in the technical model of intra-arterial delivery, the rabbit VX2 liver cancer model, to determine tumor retention and macroscale dosimetry. The αRPT-LIPIODOL® emulsions demonstrating superior therapeutic efficacy in the mouse models, and superior retention in the rabbit model will be moved forward for MTD studies and therapeutic efficacy studies using the technical model of intra-arterial delivery (rabbit model).

15.1 Mouse Studies.

15.1.1 Therapeutic efficacy. The presently disclosed αRPT-LIPIODOL® emulsions will be assessed by both histological analysis of damage to the tumors, surrounding tissue, and selected organ tissue, as well as by monitoring tumor regression over the course of 120 d following the initial treatment. Histological analysis will be performed for each group when one of the end-point conditions are met, specifically apoptosis and necrosis will be monitored in the excised tumors, as well as damage to the kidneys and liver. The αRPT-LIPIODOL® treatment demonstrating higher therapeutic efficacy in the mouse models will be moved forward to the rabbit studies.

15.1.2 Treatment Groups. Mice (4-6 weeks, n=15/group) bearing HEP2G tumors and Balb/c mice bearing 4T1 tumors will be initially randomly separated into 4 groups outlined immediately herein below. The therapeutic dose of αRPT-LIPIODOL® will be based on previously calculated MTD for αRPT. Mice bearing subcutaneous tumors (50-100 mm³) will be injected intratumorally with one of the following treatment groups:

1. Group 1 will serve as a non-treated control group. Mice will receive a single injection of saline.

2. Group 2 will serve as a control to support the combined use of αRPT and LIPIODOL®. Mice will receive a single injection of LIPIODOL® without the αRPT.

3. Group 3 will serve as a control to support the combined use of αRPT and LIPIODOL®. Mice will receive a single dose αRPT (15 kBq) without LIPIODOL®.

4. Group 4 will serve as the combined treatment of αRPT-LIPIODOL®. Mice will receive a single injection of the αRPT-LIPIODOL® (15 kBq).

Mice will be monitored until one of the following end-point conditions is met:

1. 20% loss of body mass;

2. Mice showing signs of pain and/or distress;

3. Tumors reaching 1000 mm³ as determined by volume=0.5 (Length×Width²); or 4. 6 months post-treatment.

15.2 Rabbit Studies.

The hepatic VX2 rabbit tumor model will provide an animal model to evaluate the clinically relevant intra-arterial injections of LIPIODOL® emulsions.

15.2.1. SPECT Imaging. Liver VX-2 bearing rabbits (n=3) injected via the hepatic artery will be evaluated by SPECT imaging with the selected ¹¹¹In-DOTA-alkyl-LIPIODOL® emulsion and ¹¹¹In-DOTAGA-TDA as determined by therapeutic efficacy within the mouse models. SPECT imaging will be performed at 2 h; 1 day, 3 days, 6 days, 9 days, 12 days and 15 days post-injection. Tumor uptake and retention will be determined through analysis of SPECT images as compared to a known standard, as well as uptake and retention in selected organs, including blood, heart, lung, liver, kidneys, spleen, small and large intestine, muscle, and bone. Blood will also be collect prior to injection. In addition, to uptake and clearance in the blood will be prepared and injected onto a HPLC to assess the in vivo stability of the αRPT. The length to monitor tumor uptake and retention in vivo (15 days) is approximately 5 half-lives of ¹¹¹In. The remaining activity at the latter time points may not be sufficient for SPECT imaging due to decay and biological clearance. SPECT images will be collected over a 6-day window, providing sufficient data for dosimetric calculations. However, if image and not able to collected out to 15 days, ex vivo biodistributions will be conducted with the ²²⁵Ac-labeled (T_{1/2}=10 days) αRPT-LIPIODOL®; the long half-life of ²²⁵Ac will allow us to monitor distribution and retention over the proposed 15 day-window. Furthermore, the potential of quantitative SPECT reconstruction methods to imaging ²²⁵Ac-labeled αRPT also will be explored, potentially helping to address the low activity of the companion imaging agents at the latter time points.

15.2.2. Dosimetry will be performed to calculate an acceptable, safe and effective dose of administered activities from the SPECT imaging rabbit data. The estimated dose will be evaluated in the proposed MTD studies. In addition to the use of micro-scale dosimetry described hereinabove, MTDs for α-particles need to account for the relative biological effect (RBE). A new standard has been established for RBE reporting based on the ICRU standard dose reporting EQD2, named RBE2, which eliminates dose and reference dependency and is immediately translatable to other studies and contexts. From the MTDs calculated and comparisons to traditional radiation MTDs, the RBE2 values for normal organs will be established.

15.2.3. MTD studies. The 3+3 design (Fibonacci mathematical series) will be used for identifying the MTD, minimizing the number of animals exposed to toxic doses. Three non-tumor bearing animals will be enrolled at dose I based on the dosimetry data. If one dose limiting toxicity (DLT) is encountered, three more animals will be enrolled at dose I+1. If one DLT is observed, three additional rabbits will be treated at this level with dose escalation only if no additional DLTs. If ≥2 DLTs, prior dose level is defined as MTD. The MTD is decided when six animals are treated at a dose level with <2 DLTs. The Continual Reassessment Method (CRM) and Bayesian Logistic Regression Method (BLRM) will be used to minimize the number of animals enrolled in this cohort.

15.2.4. Therapeutic efficacy. The therapeutic efficacy of selected αRPT-LIPIODOL® will be assessed by both histological analysis of damage to the tumors, surrounding tissue, and select organ tissue, as well as by monitoring tumor regression by CT imaging over the course of 120 d following the initial treatment. Histological analysis will be performed for each group (n=5) when one of the end-point conditions is met, specifically apoptosis and necrosis will be monitored in the excised tumors.

15.2.5. Biodistribution Studies. Liver VX-2 bearing rabbits will be injected via the hepatic artery with a treatment outlined under the Treatment Groups immediately herein below.

15.2.5.1. Treatment Groups. The therapeutic dose of αRPT-LIPIODOL® will be at the activity determined by the MTD studies. Rabbits presenting with VX2-tumors (n=12) will be injected intratumorally with one of the following treatment groups:

1. Group 1 will serve as a non-treated control group. Rabbits will receive a single injection of saline.

2. Group 2 will serve as the combined treatment of αRPT-LIPIODOL®.

Rabbits will receive a single injection of the αRPT-LIPIODOL® (15 kBq).

Rabbits will be monitored until one of the following end-point conditions is met:

1. 20% loss of body mass;

2. Rabbits showing signs of pain and/or distress; or

3. 6 Months Post-Treatment.

15.2.5.2. Sample size calculations and statistical analysis: Sample size calculations in the treatment groups are based on the assumption that mean percent tumor necrosis in the treatment group is 80 (SD=10) and in the control (PBS) group 65 (SD=10). Assuming significance level of 0.05 and power of 0.9, 10 animals are needed per group and for a single time point of euthanasia. In addition to CRN and BLRM, statistical analysis will include t-tests for paired comparisons, Kaplan-Meier survival analysis and log-rank tests (with p-values≤0.05).

### Example 16

### Delivery of ¹¹¹In-Labeled LIPIODOL® Emulsion to Tumor in Mouse Model

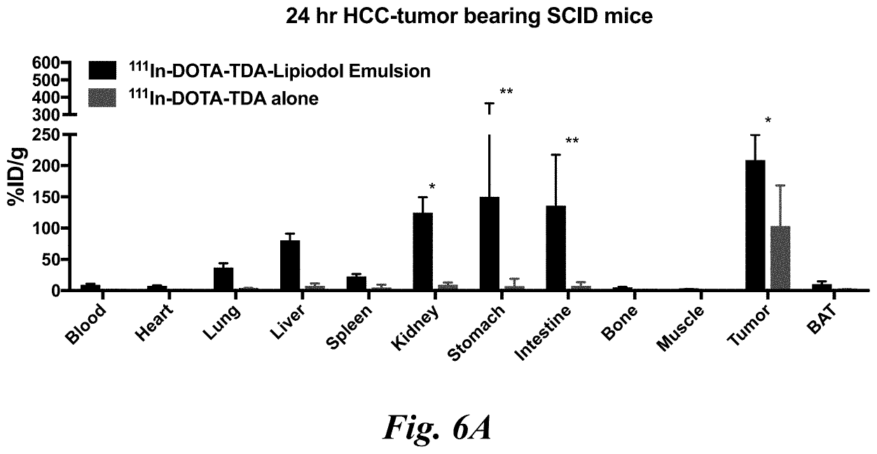

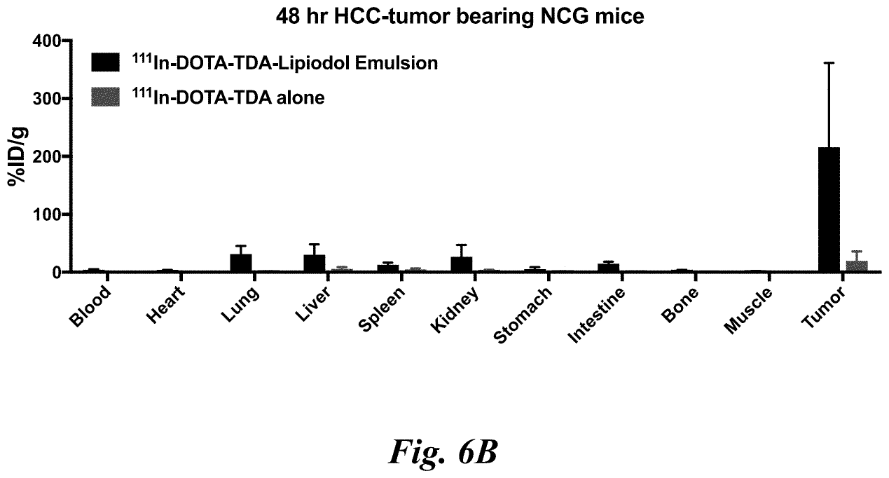

¹¹¹In-DOTA-tetradecylamine(TDA) was synthesized with a radiolabeling yield >95%. The retention of ¹¹¹In-DOTA-TDA within a hepatocellular carcinoma mouse model was evaluated; Hep G2 tumor-bearing SCID mice (24 hours, n=3) and NCG mice (48 hours, n=3) following intra-tumoral injections of ¹¹¹In-DOTA-TDA alone or as a Lipiodol emulsion (FIG. 6). A 2-way ANOVA test demonstrated that the ¹¹¹In-DOTA-TDA-LIPIODOL® emulsion had significantly higher uptake within the tumor as compared to ¹¹¹In-DOTA-TDA alone after 24 hours (209±39.7% ID/g vs. 103±65.0% ID/g; p≤0.05) and 48 hours (216±145% ID/g vs. 20.0±15.9% ID/g; p≤0.0001), supporting the concept that LIPIODOL® is able to retain the described agents.

### Example 17

### Octanol Partiation Coefficients of ¹¹¹In- and ²²⁵Ac-Labeled DOTA-TDA and ¹¹¹In-DOTA-octadecylamine(ODA)

¹¹¹In-DOTA-TDA (>95%), ²²⁵AcDOTA-TDA (87.7%), and ¹¹¹In-DOTA-ODA (85.1%) were synthesized. A greater than 95% purity was obtained following purification with a Seppak. The following Log P_{oct} were determined: 1.57±0.005 for ¹¹¹In-DOTA-TDA, 1.33±0.01 for ²²⁵Ac-DOTA-TDA, and 1.78±0.02 for ¹¹¹In-DOTA-ODA.

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Ahmadzadehfar, H., et al., The significance of 99mTc-MAA SPECT/CT liver perfusion imaging in treatment planning for 90Y-microsphere selective internal radiation treatment. J Nucl Med, 2010. 51(8): p. 1206-12.
Attaluri, A., et al., Image-guided thermal therapy with a dual-contrast magnetic nanopartide formulation: A feasibility study. Int J Hyperthermia, 2016. 32(5): p. 543-57.
Azcan, N., Ozturk Kalender, B., and Kara, M., Investigation of Turkish Poppy Seeds and Seed Oils. Chemistry of Natural Compounds, 2004. 40: p. 370.
Ballangrud, A. M., et al., Alpha-Particle Emitting Atomic Generator (Actinium-225)-Labeled Trastuzumab (Herceptin) Targeting of Breast Cancer Spheroids: Efficacy versus HER2/neu Expression. Clin Cancer Res, 2004. 10(13): p. 4489-4497.
Ballangrud, A. M., et al., Relative efficacy of the alpha particle emitters Bi-213 and Ac-225 for radioimmunotherapy against micrometastases. Proc AACR, 2000. 41: p. 289.
Ballangrud, A. M., et al., Response of LNCaP spheroids after treatment with an alpha-particle emitter (213Bi)-labeled anti-prostate-specific membrane antigen antibody (J591). Cancer Res, 2001. 61(5): p. 2008-14.
Barendsen, G. W. and A. J. Vergroesen, Irradiation of Human Cells in Tissue Culture with Alpha-Rays, Beta-Rays and X-Rays. International Journal of Radiation Biology and Related Studies in Physics Chemistry and Medicine, 1960a. 2(4): p. 441-441.
Barendsen, G. W., et al., Effect of Different Ionizing Radiations on Human Cells in Tissue Culture 0.2. Biological Experiments. Radiation Research, 1960b. 13(6): p. 841-849.
Beaino, W., J. R. Nedrow, and C. J. Anderson, Evaluation of (68)Ga-and (177)Lu-DOTA-PEG4-LLP2A for VLA-4-Targeted PET Imaging and Treatment of Metastatic Melanoma. Mol Pharm, 2015. 12(6): p. 1929-38.
Bloomer, W. D., et al., 211At radiocolloid therapy: further observations and comparison with radiocolloids of 32P, 165Dy, and 90Y. Int.J Radiat.Oncol.Biol.Phys., 1984. 10(3): p. 341-348.
Bloomer, W. D., et al., Astatine-211—tellurium radiocolloid cures experimental malignant ascites. Science, 1981. 212(4492): p. 340-341.
Bloomer, W. D., et al., Therapeutic applications of Auger and alpha emitting radionuclides. Strahlentherapie., 1984. 160(12): p. 755-757.
Decristoforo, C., et al., 68Ga-and 111In-labelled DOTA-RGD peptides for imaging of alphavbeta3 integrin expression. Eur J Nucl Med Mol Imaging, 2008. 35(8): p. 1507-15.
Gholamrezanezhad, A., et al., Evaluation of 70-150-mum doxorubicin-eluting beads for transcatheter arterial chemoembolization in the rabbit liver VX2 tumour model. Eur Radiol, 2016. 26(10): p. 3474-82.
Hind, R. E., et al., Biodistribution of Lipiodol following hepatic arterial injection. Br J Surg, 1992. 79(9): p. 952-4.
Hobbs, R., et al., A nephron-based and cell-based model of renal toxicity for a-particle radiopharmaceutical therapy. Phys Med Biol, 2012a. 57(13): p. 4403-24.
Hobbs, R., McGuffie, A., Plyku, D, and Sgouros, G., Micro-scale Modeling for the Salivary Gland: Insights into Toxicity from 1311 Therapy for Thyroid Cancer, in EANM. 2017: Vienna.
Hobbs, R. F., et al., A bone marrow toxicity model for (2)(2)(3)Ra alpha-emitter radiopharmaceutical therapy. Phys Med Biol, 2012b. 57(10): p. 3207-22.
Hobbs, R. F., et al., A model of cellular dosimetry for macroscopic tumors in radiopharmaceutical therapy. Med Phys, 2011. 38(6): p. 2892-903.
Hogberg, J., Rizell, M., Hultborn, R., Svensson, J., Henrikson, O., Moine, J., Gjertsson, P., and Bernhardt, P., Microsphere distributions and small-scale dosimetry in selective internal radiation therapy. J Nucl Med, 2015. 56(3): p. 285.
Humm, J. L. and L. M. Chin, A model of cell inactivation by alpha particle internal emitters. Radiat Res, 1993. 134(2): p. 143-50.
Humm, J. L., A microdosimetric model of astatine-211 labeled antibodies for radioimmunotherapy. Int J Radiat Oncol Biol Phys, 1987. 13(11): p. 1767-73.
Imam, S. K., Advancements in cancer therapy with alpha-emitters: a review. Int.J Radiat.Oncol Biol.Phys., 2001. 51(1): p. 271-278.
Josefsson, A., et al., Small Scale Renal Dosimetry for Alpha Particle Radiopharmaceutical Therapy of Metastatic Breast Cancer With ²²⁵Ac-7.16.4. International Journal of Radiation Oncology⋅Biology ⋅Physics, 2016. 93(3): p. S149-S150.
Josefsson, A., Kiess, A. P., Min, I. I., Back, T., Sgouros, G., and Hobbs, R. F. Combined Model-Based and Patient-Specific Dosimetry for Risk Evaluation of 18F-DCFPyL, a PSMA-Targeted PET Agent for Patients with Metastatic Prostate Cancer (PCa).” in ASTRO. 2017. San Antonio, Tex.
Kan, Z. and D.C. Madoff, Liver anatomy: microcirculation of the liver. Semin Intervent Radiol, 2008. 25(2): p. 77-85.
Kassis, A. I., et al., The Invitro Radiobiology of Astatine-211 Decay. Radiation Research, 1986. 105(1): p. 27-36.
Kennedy, A. S., et al., Resin 90Y-microsphere brachytherapy for unresectable colorectal liver metastases: modern USA experience. Int J Radiat Oncol Biol Phys, 2006. 65(2): p. 412-25.
Kidd, J. G. and P. Rous, A Transplantable Rabbit Carcinoma Originating in a Virus-Induced Papilloma and Containing the Virus in Masked or Altered Form. J Exp Med, 1940. 71(6): p. 813-38.
Kozak, R. W., et al., Bismuth-212-Labeled Anti-Tac Monoclonal-Antibody-Alpha-Particle-Emitting Radionuclides As Modalities for Radioimmunotherapy. Proceedings of the National Academy of Sciences of the United States of America, 1986. 83(2): p. 474-478.
Kurtzman, S. H., et al., Bismuth-212 Linked to An Antipancreatic Carcinoma Antibody-Model for Alpha-Particle-Emitter Radioimmunotherapy. J Natl Cancer Inst, 1988. 80(6): p. 449-452.
Lee, K. H., et al., Considerations for implantation site of VX2 carcinoma into rabbit liver. J Vasc Intery Radiol, 2009a. 20(1): p. 113-7.
Lee, K. H., et al., Distribution of iron oxide-containing Embosphere particles after transcatheter arterial embolization in an animal model of liver cancer: evaluation with MR imaging and implication for therapy. J Vasc Intery Radiol, 2008. 19(10): p. 1490-6.
Lee, K. H., et al., Doxorubicin-loaded QuadraSphere microspheres: plasma pharmacokinetics and intratumoral drug concentration in an animal model of liver cancer. Cardiovasc Intervent Radiol, 2010. 33(3): p. 576-82.
Lee, K. H., et al., Evaluation of different calibrated spherical polyvinyl alcohol microspheres in transcatheter arterial chemoembolization: VX2 tumor model in rabbit liver. J Vasc Intery Radiol, 2008. 19(7): p. 1065-9.
Lee, K. H., et al., Percutaneous US-guided implantation of Vx-2 carcinoma into rabbit liver: a comparison with open surgical method. J Surg Res, 2009b. 155(1): p. 94-9.
Leung, W. T., et al., Measuring lung shunting in hepatocellular carcinoma with intrahepatic-arterial technetium-99m macroaggregated albumin. J Nucl Med, 1994. 35(1): p. 70-3.
Liapi, E. and J. F. Geschwind, Chemoembolization for primary and metastatic liver cancer. Cancer J, 2010. 16(2): p. 156-62.
Liapi, E. and J. F. Geschwind, Transcatheter and ablative therapeutic approaches for solid malignancies. J Clin Oncol, 2007. 25(8): p. 978-86.
Liapi, E., et al., Assessment of tumoricidal efficacy and response to treatment with 18F-FDG PET/CT after intraarterial infusion with the antiglycolytic agent 3-bromopyruvate in the VX2 model of liver tumor. J Nucl Med, 2011. 52(2): p. 225-30.
Llovet, J. M., et al., Arterial embolisation or chemoembolisation versus symptomatic treatment in patients with unresectable hepatocellular carcinoma: a randomised controlled trial. Lancet, 2002. 359(9319): p. 1734-9.
Lub-de Hooge, M. N., et al., Preclinical characterisation of 111In-DTPA-trastuzumab. Br J Pharmacol, 2004. 143(1): p. 99-106.
Macklis, R. M., et al., Radioimmunotherapy with Alpha-Particle Emitting Immunoconjugates. Science, 1988. 240(4855): p. 1024-1026.
Maluccio, M. A., et al., Transcatheter arterial embolization with only particles for the treatment of unresectable hepatocellular carcinoma. J Vasc Intery Radiol, 2008. 19(6): p. 862-9.
Marelli, L., et al., Transarterial injection of (131)I-lipiodol, compared with chemoembolization, in the treatment of unresectable hepatocellular cancer. J Nucl Med, 2009. 50(6): p. 871-7.
McDevitt, M. R., et al., Radioimmunotherapy with alpha-emitting nuclides. Eur J Nucl Med, 1998. 25(9): p. 1341-51.
McDevitt, M. R., et al., Tumor therapy with targeted atomic nanogenerators. Science, 2001. 294(5546): p. 1537-40.
Milenic, D. E., et al., Potentiation of high-LET radiation by gemcitabine: targeting HER2 with trastuzumab to treat disseminated peritoneal disease. Clin Cancer Res, 2007. 13(6): p. 1926-35.
Nedrow, J. R., et al., Positron emission tomographic imaging of copper 64-and gallium 68-labeled chelator conjugates of the somatostatin agonist tyr 3-octreotate. Mol Imaging, 2014. 13.
Nilsson, S., et al., Bone-targeted radium-223 in symptomatic, hormone-refractory prostate cancer: a randomised, multicentre, placebo-controlled phase II study. Lancet Oncol, 2007. 8(7): p. 587-94.
Nilsson, S., et al., Clinical Experience and Radiation Safety of the First-in-Class Alpha-Pharmaceutical, Alpharadin (radium-223) in Patients with Castration-Resistant Prostate Cancer (CRPC) and Bone Metastases. International Journal of Radiation Oncology*Biology*Physics, 2010. 78(3, Supplement 1): p. S375-S376.
Parvinian, A., L. C. Casadaban, and R. C. Gaba, Development, growth, propagation, and angiographic utilization of the rabbit VX2 model of liver cancer: a pictorial primer and “how to” guide. Diagn Intery Radiol, 2014. 20(4): p. 335-40.
Pfost, B., et al., Intravesical alpha-radioimmunotherapy with 213Bi-anti-EGFR-mAb defeats human bladder carcinoma in xenografted nude mice. J Nucl Med, 2009. 50(10): p. 1700-8.
Raju, M. R., et al., Radiobiology of Alpha-Particles 0.3. Cell Inactivation by Alpha-Particle Traversals of the Cell-Nucleus. Radiation Research, 1991. 128(2): p. 204-209.
Rosenblat, T. L., et al., Phase I trial of the targeted alpha particle nano-generator actinium-225 (Ac-225)-HuM195 (Anti-CD33) in acute myeloid leukemia (AML). Blood, 2007. 110(11): p. 910.
Rosenblat, T. L., et al., Sequential cytarabine and alpha particle immunotherapy with bismuth-213-lintuzumab (HuM195) for acute myeloid leukemia. Clin Cancer Res, 2010. 16(21): p. 5303-11.
Sgouros, G., Alpha particles for targeted therapy. Adv Drug Deliv Rev, 2008. 60(12): p. 1402-6.
Sgouros, G., et al., Pharmacokinetics and dosimetry of an alpha particle emitter labeled antibody: 213Bi-HuM195 (anti-CD33) in patients with leukemia. J Nucl Med, 1999. 40(11): p. 1935-46.
Vali, M., et al., Intraarterial therapy with a new potent inhibitor of tumor metabolism (3-bromopyruvate): identification of therapeutic dose and method of injection in an animal model of liver cancer. J Vasc Intery Radiol, 2007. 18(1 Pt 1): p. 95-101.
Vali, M., et al., Targeting of VX2 rabbit liver tumor by selective delivery of 3-bromopyruvate: a biodistribution and survival study. J Pharmacol Exp Ther, 2008. 327(1): p. 32-7.
Vogl, T. J., et al., Colorectal liver metastases: regional chemotherapy via transarterial chemoembolization (TACE) and hepatic chemoperfusion: an update. Eur Radiol, 2007. 17(4): p. 1025-34.
Vossen, J. A., et al., Diffusion-weighted and Gd-EOB-DTPA-contrast-enhanced magnetic resonance imaging for characterization of tumor necrosis in an animal model. J Comput Assist Tomogr, 2009. 33(4): p. 626-30.
Yang, T. X., T. C. Chua, and D. L. Morris, Radioembolization and chemoembolization for unresectable neuroendocrine liver metastases—a systematic review. Surg Oncol, 2012. 21(4): p. 299-308.
Zalutsky, M. R., et al., Clinical Experience with {alpha}-Particle Emitting 211At: Treatment of Recurrent Brain Tumor Patients with 211At-Labeled Chimeric Antitenascin Monoclonal Antibody 8106. J Nucl Med, 2008. 49(1): p. 30-38.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A formulation comprising a chelator coupled to a lipophilic alkyl side chain and an emulsifying agent comprising poppy seed oil.

2. The formulation of claim 2, wherein the chelator is selected from the group consisting of: H₂macropa, 2-(4-isothiocyanatobenzyl)-1,4,7,10,13,16-hexaazacyclohexadecane-1,4,7,10,13,16-hexaacetic acid (HEHA-NCS), 1,4,7,10,13,16-hexaazacyclohexadecane-N,N′,N″,N′″,N″,N′″″-hexaacetic acid (HEHA),

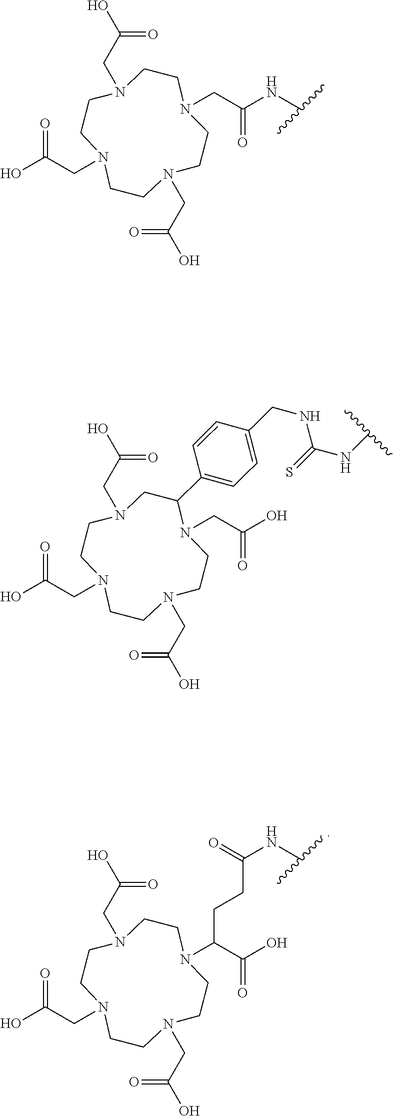 [SMILES: *NC(=O)CN1CCN(CC(=O)O)CCN(CC(=O)O)CCN(CC(=O)O)CC1 ; *NC(=S)NCc1ccc(C2CN(CC(=O)O)CCN(CC(=O)O)CCN(CC(=O)O)CCN2CC(=O)O)cc1 ; *NC(=O)CCC(C(=O)O)N1CCN(CC(=O)O)CCN(CC(=O)O)CCN(CC(=O)O)CC1]

3. The formulation of claim 1, wherein the alkyl side chain comprises a fatty acid.

4. The formulation of claim 3, wherein the fatty acid is selected from a saturated fatty acid and an unsaturated fatty acid.

5. The formulation of claim 4, wherein the saturated fatty acid is selected from the group consisting of caprylic acid, capric acid, lauric acid, myristic acid, palmitic acid, stearic acid, arachidic acid, behenic acid, lignoceric acid, and cerotic acid.

6. The formulation of claim 5, wherein the unsaturated fatty acid is selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, and docosahexaenoic acid.

7. The formulation of claim 1, wherein the alkyl side chain is selected from the group consisting of:

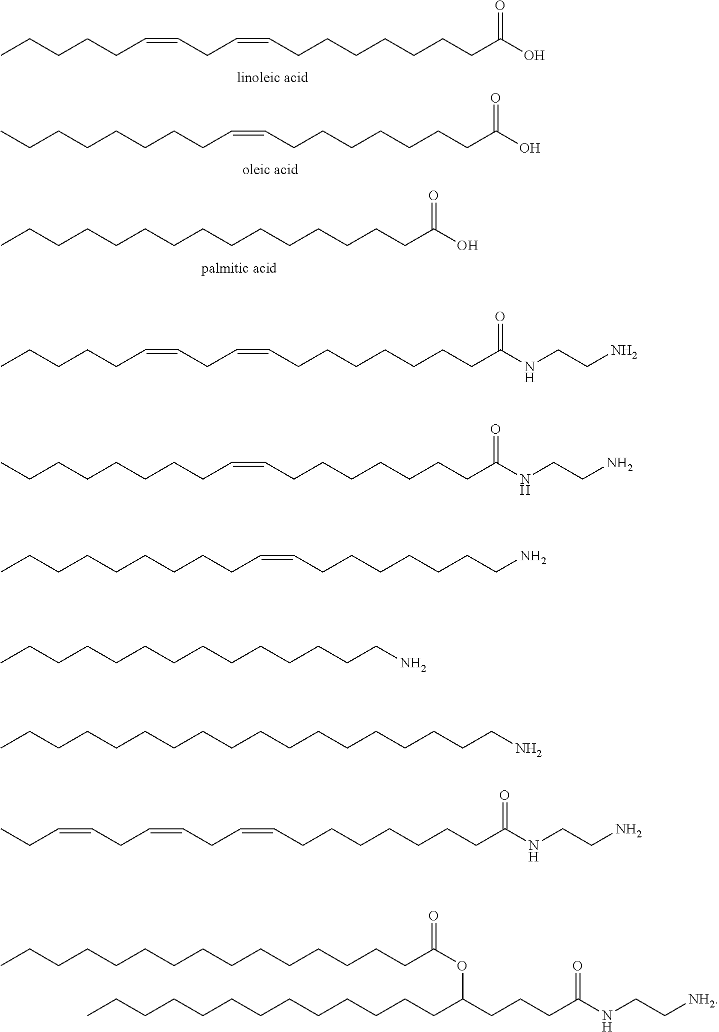

8. The formulation of claim 1, wherein the chelator is coupled to the alkyl side chain through an amine-reactive pendant group on the carbon backbone of the alkyl side chain or through an activated carboxylic acid of the alkyl side chain.

9. The formulation of claim 1, wherein the poppy seed oil comprises about 50% to about 75% linoleic acid, about 10% to about 30% oleic acid, and about 5% to about 25% palmitic acid.

10. The formulation of claim 1, wherein the poppy seed oil comprises about 55% to about 70% linoleic acid, about 15% to about 20% oleic acid, and about 10% to about 20% palmitic acid

11. The formulation of claim 1, wherein the chelator coupled to the lipophilic alkyl side chain has the following chemical structure:

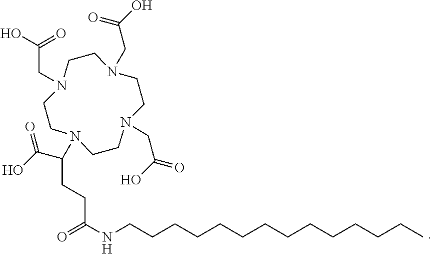 [SMILES: CCCCCCCCCCCCCCNC(=O)CCC(C(=O)O)N1CCN(CC(=O)O)CCN(CC(=O)O)CCN(CC(=O)O)CC1]

12. The formulation of claim 1 further comprising an alpha-emitting radionuclide chelated to the chelator through one or more coordinate bonds.

13. The formulation of claim 12, wherein the alpha-emitting radionuclide is selected from the group consisting of Astatine-211 (²¹¹At), Bismuth-212 (²¹²Bi), Bismuth-213 (²¹³Bi), Actinium-225 (²²⁵Ac), Radium-223 (²²³Ra), Lead-212 (²¹²Pb), Thorium-227 (²²⁷Th), and Terbium-149 (¹⁴⁹Tb).

14. The formulation of claim 13, wherein the alpha-emitting radionuclide is ²²⁵Ac.

15. The formulation of claim 1 further comprising a radionuclide suitable for use in single-photon emission computed tomography (SPECT) imaging or positron emission tomography (PET) imaging, wherein the radionuclide is chelated to the chelator through one or more coordinate bonds.

16. The formulation of claim 15, wherein the radionuclide suitable for use in SPECT imaging or PET imaging is selected from the group consisting of indium-111 (¹¹¹In), technetium-99m (^{99m}Tc), thallium-201 (²⁰¹Tl), terbium-155 (¹⁵⁵Tb), gallium-68 (⁶⁸Ga), copper-64 (⁶⁴Cu), and zirconium-89 (⁸⁹Zr).

17. A method for treating cancer in a subject in need of treatment thereof, the method comprising administering to the subject an effective amount of a formulation of any of claims 1-14.

18. The method of claim 17, wherein the cancer comprises a primary liver cancer.

19. The method of claim 18, wherein the primary liver cancer comprises hepatocellular carcinoma (HCC).

20. The method of claim 17, wherein the cancer comprises a metastatic cancer.

21. The method of claim 20, wherein the metastatic cancer comprises metastatic breast cancer.

22. The method of claim 17, wherein the cancer comprises a lymphatic cancer.

23. The method of claim 17, wherein the formulation is delivered intra-arterially.

24. A method for imaging a cancer in a subject, the method comprising administering to the subject the formulation of claim 15 or 16 and taking an image.

25. The method of claim 24, wherein the imaging is SPECT imaging.

26. The method of claim 24, further comprising treating the subject with a formulation of claims 1-14.
